(19) United States
(12) Patent Application Publication
(10) Pub. No.: US 2020/0069912 A1
(43) Pub. Date: Mar. 5, 2020

(54) ADJUSTABLE CALIBER CATHETER SYSTEM

(71) Applicant: THE REGENTS OF THE UNIVERSITY OF CALIFORNIA, Oakland, CA (US)

(72) Inventor: Satoshi Tateshima, Los Angeles, CA (US)

(73) Assignee: THE REGENTS OF THE UNIVERSITY OF CALIFORNIA, Oakland, CA (US)

(21) Appl. No.: 16/569,164

(22) Filed: Sep. 12, 2019

Related U.S. Application Data
(63) Continuation of application No. PCT/US2018/022187, filed on Mar. 13, 2018.
(60) Provisional application No. 62/470,641, filed on Mar. 13, 2017.

(51) Int. Cl. A61M 25/00; A61F 2/958

(52) U.S. Cl. CPC A61M 25/0074; A61F 2/958; A61M 2025/107; A61M 2025/0042; A61M 25/0013

(57) ABSTRACT

As a clot retrieval device, a dual-layer expandable aspiration catheter having a catheter with a distal slit and a flexible sheath surrounding the slit. The dual-layer expandable aspiration catheter maintains column strength (along the axis of the catheter) while the distal slit provide radial flexibility only to circumferential direction. This structure prevents the distal expandable segment from crumpling, thereby the stent retriever can be pulled into the expandable catheter smoothly.

### CROSS-REFERENCE TO RELATED APPLICATIONS

This application claims priority to, and is a 35 U.S.C. § 111(a) continuation of, PCT international application number PCT/US2018/022187 filed on Mar. 13, 2018, incorporated herein by reference in its entirety, which claims priority to, and the benefit of, U.S. provisional patent application Ser. No. 62/470,641 filed on Mar. 13, 2017, incorporated herein by reference in its entirety. Priority is claimed to each of the foregoing applications.

The above-referenced PCT international application was published as PCT International Publication No. WO 2018/169959 on Sep. 20, 2018, which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to aspiration catheters, and more particularly to aspiration catheters for stent and/or clot retrieval.

### 2. Background Discussion

Inherently, the smaller the size of a catheter is, the more navigable it becomes. That is the reason why so many different micro-catheters (less than 3 French) are being used in the field of neuro-intervention. However, recent advancements in catheter technology have enabled a relatively large catheter navigable through the tortuous skull base vasculature, and reachable to the delicate intracranial vasculature. Those latest relatively large catheters (4-6 French), called intermediate catheters, fit in a commonly utilized guiding sheath or guiding catheter system placed in the neck. The intermediate catheter placed in the skull base or intracranial vasculature gives a strong support to the micro-catheter, which makes micro-catheter control easier and safer.

The 4-6 French sized intermediate catheters are also used as a clot aspiration device that is blocking a large vessel in the brain causing a stroke to the patient with or without a stent retriever device. Ideally, the size of a catheter should be matched to the size of the artery to maximize the clot capturing capability. However, it would require a larger guiding catheter than the current standard system (6-9 Fr) in order to be compatible with a larger intermediate catheter that matches the size of the target artery. Also, such a larger intermediate catheter that has exactly the same size of the target artery would not be so navigable due to ledge effect or friction. Given the work-flow in the acute stroke intervention where 1.9 million nerves are lost every minute and chance of good clinical recovery is diminished by 1% every 5 minutes, it would be too time consuming and virtually impossible to select a right sized intermediate catheter that perfectly fits the size of the target artery.

In a conventional aspiration/coaxial catheter, the stent retriever with clot is generally squeezed into the aspiration catheter that is smaller in caliber, with the fragments allowed to flow downstream in the blood flow causing downstream strokes.

Therefore, there is unmet need in an intermediate catheter having a distal portion that expands to the size of the target artery.

### BRIEF SUMMARY

The disclosure of the present technology is directed to a catheter that expands to the size of the target vessel by providing one or more slits to its distal tip. In order to avoid leak or to prevent a loss of aspiration performance, the distal portion of the catheter is wrapped with a sheath comprising a commonly used expandable material such as thin chronoprene, polyurethane or like compliant film.

The expandable catheter as described herein is extremely useful as an aspiration catheter for acute stroke thrombectomy. In one embodiment, a stent retriever device that expands to 4-6 mm in diameter or any equivalent device will be placed across the clot blocking the artery via the expandable catheter. Once the clot is captured by the stent retriever, the expandable catheter can be advanced and will slide over the stent retriever thus the size of the catheter expands to the size of the target artery and/or the stent retriever. In this way, the entire clot burden could be captured into the catheter without a portion of it shaved off from the stent retriever.

The expandable catheter could be as small as 3-4 French during its navigation into the intracranial vasculature therefore more navigable than 4-6 French catheters. But when needed, it can expand up to 10-12 French depending on the size of the stent retriever or equivalent device placed in the target vessel.

The utility of the expandable catheter is not just limited to the acute stroke thrombectomy. It can be used in any peripheral, body, coronary, and neuro interventions when the size of catheter has to become bigger than the size of guiding catheter system.

An aspect of the presented technology is a clot retrieval device comprising a dual-layer expandable aspiration catheter having a catheter with a distal slit and a flexible sheath surrounding the slit. An aspect of the dual layer expandable aspiration catheter is that it maintains column strength (along the axis of the catheter) while the slit(s) provide radial flexibility only to circumferential direction. This structure prevents the distal expandable segment from crumpling, thereby the stent retriever can be pulled into the expandable catheter smoothly. Having the outer expandable sheath wrapping the entire slit segment, aspiration force is not lost and also any clots coming off from the stent retriever are contained within the catheter.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 is a perspective view of a dual-layer expandable aspiration catheter in accordance with the present description.

FIGS. 2A through FIG. 2H show a method and system of using the dual-layer expandable aspiration catheter of FIG. 1 for retrieval of a clot within a body lumen.

### DETAILED DESCRIPTION

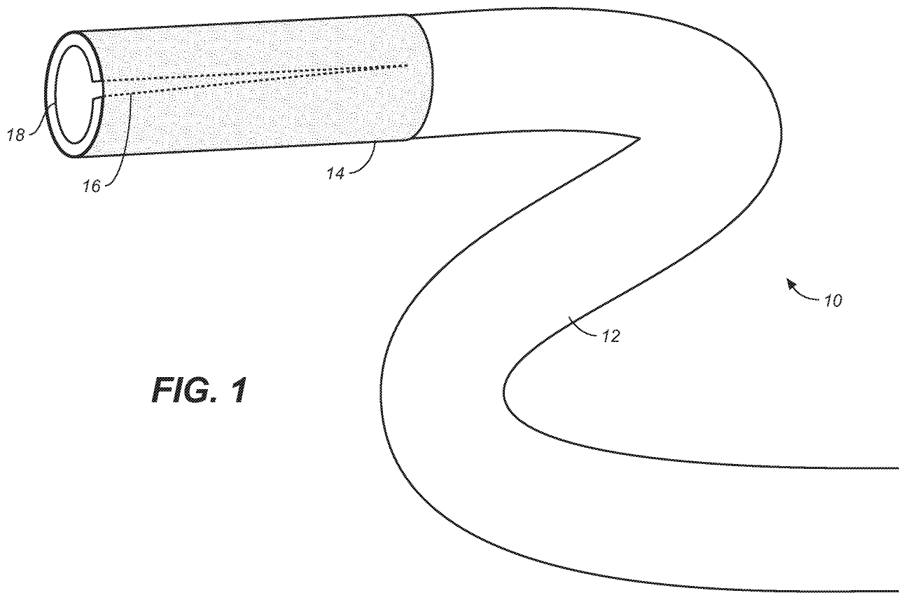

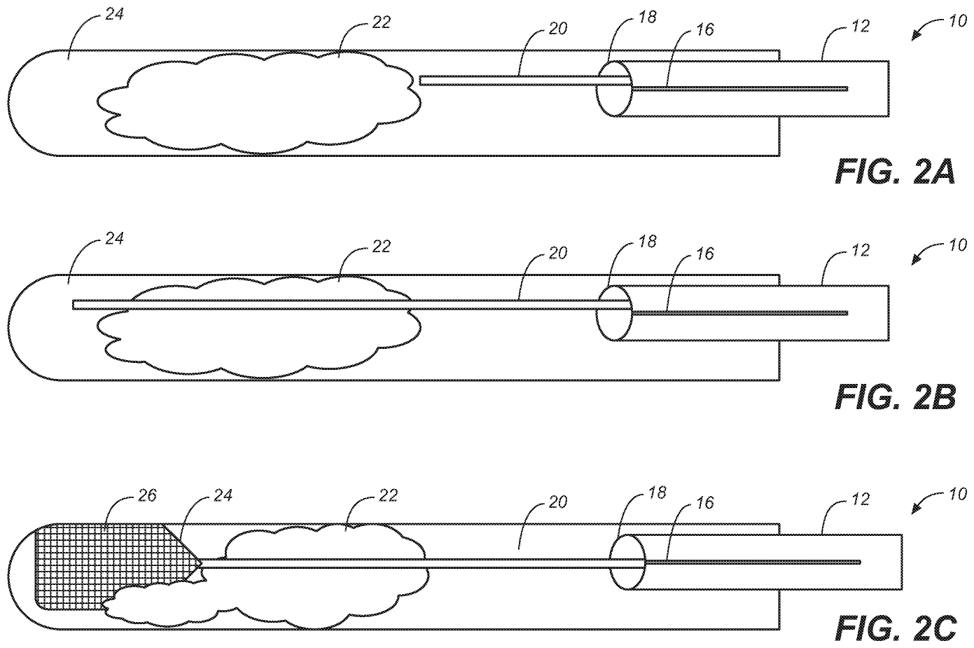

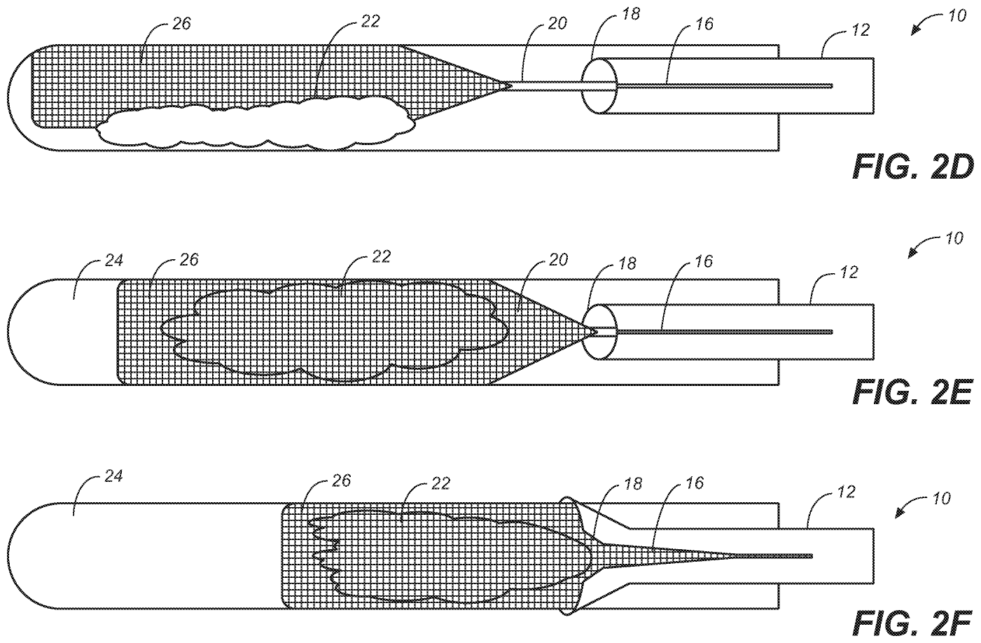

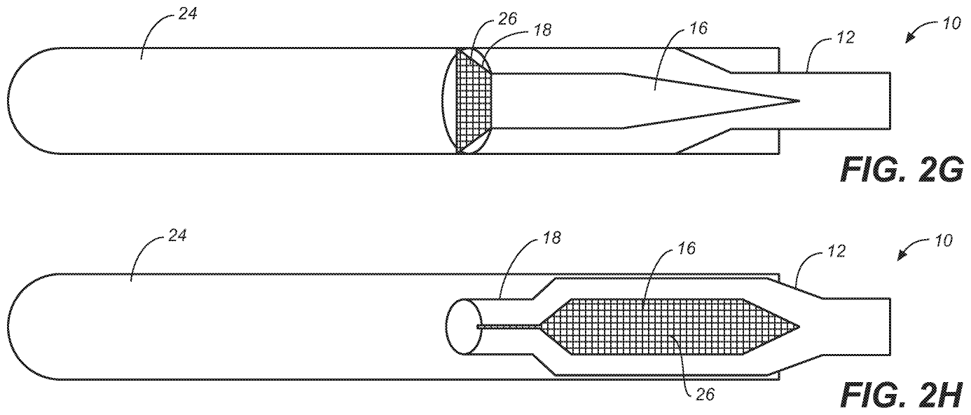

FIG. 1 shows a perspective view of a dual-layer expandable aspiration catheter 10 in accordance with the present description. The catheter 10 comprises a catheter body 12 having a single longitudinal slit 16 at the distal end 18 of the catheter body 12. The slit 16 and distal end 18 serve as a passive, expandable segment for receiving a stent/clot retriever and clot into the distal end 18 (see FIG. 2A through FIG. 2H).

A flexible sheath 14 is disposed around the expandable distal segment of the catheter 12. The sheath 14 comprises a material and diameter configured to retain the sheath 14 in a radial position snugly around the outer diameter of the catheter body 12, while allowing for radial expansion of the catheter body 12. Absent any force causing the radial expansion of the catheter 12, the sheath diameter and material are configured to passively shrink back to the outer diameter of the catheter body 12 (and promote the catheter body in doing the same).

In FIG. 1, the sheath 14 is shown as a translucent material disposed over the expandable segment of the catheter. However, it is appreciated that any flexible material may be implemented as the sheath 14, and the sheath may be disposed as a layer internal to the catheter 12. In an alternative embodiment, the sheath only covers a portion of the circumference over the slit 16, and is attached (e.g. via adhesive, staple tack or other attachment means) to the inside or outside wall of the catheter 12 to radially close off the slit 16.

FIG. 2A through FIG. 2H show a method and system of using the dual-layer expandable aspiration catheter 10 of FIG. 1 for retrieval of a clot 22 within a body lumen 24. For clarity, the sheath 14 is excluded from view in FIG. 2A through FIG. 2H. In a preferred embodiment, the lumen comprises an artery 24 with an occlusive clot burden 22. FIG. 2A shows the dual layer expandable aspiration catheter 10 placed proximal to the clot 22 in the artery 24.

In FIG. 2B, a micro-catheter 20 is advanced out distal end 18 through the clot 22 for subsequent stent retriever placement. An aspiration vacuum may be applied prior or during one or more of steps shown in FIG. 2B through FIG. 2H to help in retrieval of the clot and any potential fragments thereof.

In FIG. 2C, the stent retriever 26 is delivered through the micro-catheter 20 to the opposite side of the clot 22, where the stent retriever 26 is deployed proximally across the clot 22. further deployment of stent retriever.

FIG. 2D shows the stent retriever 26 completely deployed across the clot 22. In FIG. 2E, the stent retriever 26 is expanded radially outward to the size of the artery 24, cutting into the clot 22, with the expanded stent 26 engaging the clot burden 22 within the inner confines of the stent retriever 26.

In FIG. 2F, the stent retriever 26 and clot 22 are pulled proximally into the distal end 18 of the aspiration catheter.

Referring now to FIG. 21, as the stent retriever is pulled into the expandable aspiration catheter, the slit 16 allows the distal end 18 of the catheter to passively expand radially to the size of the stent retriever 26 or the artery 24, enabling a more complete clot capture. FIG. 2F shows the distal end 18 of the expandable aspiration catheter 10 further expanding as the stent retriever 26 is pulled into it. As shown in FIG. 2F and FIG. 2G, there is no gap between the fully expanded stent retriever 26 and aspiration catheter body 12, eliminating clot fragments being dislodged from the main clot burden 22. This action mimics a snake swallowing its prey.

Referring now to FIG. 2H, as the clot retriever 26 is completely withdrawn into the expandable catheter body 12, the distal tip 18 of the catheter body 12 converges back to the original caliber, which helps to contain the clot completely within the expandable aspiration catheter.

Eventually, the stent retriever 26 is completely withdrawn outside of the body without the need for downstream strokes.

While the single slit 16 configuration shown in FIG. 1 is a preferred configuration, it is appreciated that 2 or more slits may be positioned in spaced-apart, radial locations equally dispersed around the circumference of the distal end 18 (e.g. 180° spacing for 2 slits, 120° spacing for 3 slits, and 90° spacing for 4 slits, all not shown).

An alternative embodiment (not shown) may comprise a plurality of smaller perforating slits in place of or in combination with the one large longitudinal slit 16 disposed at the distal end of the catheter. The small slits provide a radial flexibility, but are dispersed so as to maintain columnar (axial) stiffness.

From the foregoing description, it will be appreciated that a catheter having merely a distal segment comprising of expandable material alone would generally be unable to recapture the fully opened stent retriever with the clot. Because this expandable segment is flexible in all directions, the flexible segment would be susceptible to crumpling as the stent retriever is pulled into the catheter. The crumpling works against completely capturing the clot within the stent retriever. In contrast, the dual layer expandable aspiration catheter 10 presented herein maintains columnar (i.e. axial) strength, while the slit 16 and sheath 14 provide flexibility only in the circumferential or axial direction. The structure of the dual layer expandable aspiration catheter 10, as detailed in FIG. 1, prevents distal expandable segment from crumpling, thereby the stent retriever 26 can be pulled into the expandable catheter body 12 smoothly. Having the outer expandable sheath 14 material wrapping the entire slit 16, aspiration force (e.g. from any applied vacuum) is not lost and also any clot fragments coming off from the stent retriever 26 are contained within the catheter 10.

The dual-layer expandable catheter 10 of the present description has a number of benefits. For example, the system of the present description may be implemented as a one-size catheter that fits to all intracranial arteries, thus eliminating a catheter size selection process. Furthermore, once a stent retriever with a clot is completely retrieved into the expandable catheter, the expandable catheter closes to the original size (smaller than the stent retriever's diameter). This also protects the captured clot fragments from coming out of the expandable catheter.

From the description herein, it will be appreciated that the present disclosure encompasses multiple embodiments which include, but are not limited to, the following:

1. A clot retrieval device, comprising: a catheter having a distal end configured for receiving a stent retriever having a diameter larger than a diameter of the catheter; a flexible sheath disposed in or around the catheter; the catheter comprising a longitudinal slit extending from the distal end and proximally along a portion of the catheter; said longitudinal slit providing radial flexibility while maintaining axial stiffness of the catheter; said catheter and flexible sheath configured to radially expand to accept the stent retriever.

2. The apparatus or method of any preceding or subsequent embodiment, wherein the catheter comprises a single longitudinal slit.

3. The apparatus or method of any preceding or subsequent embodiment, wherein the catheter comprises a plurality of equally-spaced longitudinal slits extending from the distal end.

4. The apparatus or method of any preceding or subsequent embodiment, wherein the catheter comprises a radially rigid section proximal to the longitudinal slit.

5. The apparatus or method of any preceding or subsequent embodiment, wherein the sheath is configured to surround the catheter and comprises a compliant material having an inner diameter that matches an outer diameter of the catheter in an unexpanded state; and wherein the compliant material of the sheath is configured to passively expand as a result of forces applied from the catheter as the catheter expands at the distal slit.

6. The apparatus or method of any preceding or subsequent embodiment, wherein diameter of the sheath promotes radial retraction of the catheter upon passing of the stent retriever.

7. The apparatus or method of any preceding or subsequent embodiment, further comprising: a microcatheter configured to house the stent retriever in a collapsed configuration and deliver the stent retriever to a location within a lumen.

8. A method of retrieving clot within a body lumen, comprising: delivering a catheter to a location in a lumen, said location comprising a clot; the catheter comprising a distal end configured for receiving a stent retriever; the catheter comprising a longitudinal slit extending from the distal end and proximally along a portion of the catheter; the catheter further comprising a flexible sheath disposed in or around the catheter; the longitudinal slit providing radial flexibility while maintaining axial stiffness of the catheter; delivering a microcatheter configured to house the stent retriever in a collapsed configuration to the location within a lumen; expanding the stent retriever at the clot; withdrawing the stent retriever and clot back into the distal end of the catheter; wherein the catheter and flexible sheath configured to radially expand to accept the stent retriever.

9. The apparatus or method of any preceding or subsequent embodiment, wherein the catheter comprises a single longitudinal slit.

10. The apparatus or method of any preceding or subsequent embodiment, wherein the catheter comprises a radially rigid section proximal to the longitudinal slit.

11. The apparatus or method of any preceding or subsequent embodiment, wherein the sheath is configured to surround the catheter and comprises a compliant material having an inner diameter that matches an outer diameter of the catheter in an unexpanded state; and wherein the sheath passively expands as a result of forces applied from the catheter as the catheter expands at the distal slit.

12. The apparatus or method of any preceding or subsequent embodiment, wherein diameter of the sheath promotes radial retraction of the catheter upon passing of the stent retriever.

13. The apparatus or method of any preceding or subsequent embodiment, wherein the sheath only partially covers a portion of the catheter circumference at the location of the slit.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the term “set” refers to a collection of one or more objects. Thus, for example, a set of objects can include a single object or multiple objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A clot retrieval device, comprising:
a catheter having a distal end configured for receiving a stent retriever having a diameter larger than a diameter of the catheter;
a flexible sheath disposed in or around the catheter;
the catheter comprising a longitudinal slit extending from the distal end and proximally along a portion of the catheter;
said longitudinal slit providing radial flexibility while maintaining axial stiffness of the catheter; and
said catheter and flexible sheath configured to radially expand to accept the stent retriever.

2. The clot retrieval device of claim 1, wherein the catheter comprises a single longitudinal slit.

3. The clot retrieval device of claim 1, wherein the catheter comprises a plurality of equally-spaced longitudinal slits extending from the distal end.

4. The clot retrieval device of claim 1, wherein the catheter comprises a radially rigid section proximal to the longitudinal slit.

5. The clot retrieval device of claim 2:
wherein the sheath is configured to surround the catheter and comprises a compliant material having an inner diameter that matches an outer diameter of the catheter in an unexpanded state; and
wherein the compliant material of the sheath is configured to passively expand as a result of forces applied from the catheter as the catheter expands at the distal slit.

6. The clot retrieval device of claim 5:
wherein diameter of the sheath promotes radial retraction of the catheter upon passing of the stent retriever.

7. The clot removal device of claim 2:
wherein the catheter has a circumference; and
wherein the sheath only partially covers a portion of the catheter circumference at the location of the slit.

8. The clot removal device of claim 2, further comprising:
a microcatheter configured to house the stent retriever in a collapsed configuration and deliver the stent retriever to a location within a lumen.

9. A method of retrieving clot within a body lumen, comprising:
delivering a catheter to a location in a lumen, said location comprising a clot;
the catheter comprising a distal end configured for receiving a stent retriever;
the catheter comprising a longitudinal slit extending from the distal end and proximally along a portion of the catheter;
the catheter further comprising a flexible sheath disposed in or around the catheter;
the longitudinal slit providing radial flexibility while maintaining axial stiffness of the catheter;
delivering a microcatheter configured to house the stent retriever in a collapsed configuration to the location within a lumen;
expanding the stent retriever at the clot;
withdrawing the stent retriever and clot back into the distal end of the catheter; and
wherein the catheter and flexible sheath configured to radially expand to accept the stent retriever.

10. The method of claim 9, wherein the catheter comprises a single longitudinal slit.

11. The method of claim 9, wherein the catheter comprises a radially rigid section proximal to the longitudinal slit.

12. The method of claim 9:
wherein the sheath is configured to surround the catheter and comprises a compliant material having an inner diameter that matches an outer diameter of the catheter in an unexpanded state; and
wherein the sheath passively expands as a result of forces applied from the catheter as the catheter expands at the distal slit.

13. The method of claim 12:
wherein diameter of the sheath promotes radial retraction of the catheter upon passing of the stent retriever.

14. The method of claim 10:
wherein the catheter has a circumference; and
wherein the sheath only partially covers a portion of the catheter circumference at the location of the slit.
